(19) United States
(12) Patent Application Publication
(10) Pub. No.: US 2020/0069038 A1
(43) Pub. Date: Mar. 5, 2020

(54) KITS FOR APPLYING COSMETIC COMPOSITIONS

(71) Applicant: L'ORÉAL, Paris (FR)

(72) Inventors: Chunhua LI, Hillsborough, NJ (US); Jody Ebanks, Bloomfield, NJ (US); Kyoo Park, Leonia, NJ (US); XianZhi Zhou, Millburn, NJ (US); Kathryn Andrea Zuccarello, Franklin Park, NJ (US); Christopher Pang, New York, NY (US)

(73) Assignee: L'ORÉAL, Paris (FR)

(21) Appl. No.: 16/119,777

(22) Filed: Aug. 31, 2018

(51) Int. Cl. A46B 9/02; A61K 8/34; A61K 8/81; A61Q 1/10

(52) U.S. Cl. CPC A46B 9/021; A61K 8/34; A61K 8/8158; A61K 8/8182; A61Q 1/10; A46B 2200/1053; A61K 2800/30; A61K 2800/48; A61K 2800/522; A61K 2800/524; A61K 2800/43; A61K 2800/87

(57) ABSTRACT

Kits for applying cosmetic compositions to keratinous material, in particular to (1) applicators comprising a brush comprising a core having an outer surface and a longitudinal axis; a plurality of bristles protruding from the core and spaced apart along the longitudinal axis; and a high density zone that covers at least a portion of the outer surface of the core, the high density zone having a surface bristle density of up to 7 bristles per square millimeter of area of the outer surface of the core, and wherein the bristles have a length from about 0.6 mm to about 4 mm and (2) cosmetic compositions for application to hair, eyebrows and/or eyelashes having a viscosity of less than about 250 Pa·S when measured at a shear rate at 5 s⁻¹ are provided.

### FIELD OF THE INVENTION

The present disclosure relates generally to kits for applying cosmetic compositions to keratinous material, in particular to (1) applicators comprising a brush comprising a core having an outer surface and a longitudinal axis; a plurality of bristles protruding from the core and spaced apart along the longitudinal axis; and a high density zone that covers at least a portion of the outer surface of the core, the high density zone having a surface bristle density of up to 7 bristles per square millimeter of area of the outer surface of the core, and wherein the bristles have a length from about 0.6 mm to about 4 mm and (2) cosmetic compositions for application to hair, eyebrows and/or eyelashes having a viscosity of less than about 250 Pa·S when measured at a shear rate at 5 s⁻¹.

### BACKGROUND OF THE INVENTION

Mascara compositions are commonly used to enhance the appearance of eyelashes. Conventional mascara compositions generally use waxes to form crystalline network structures to enhance curl, volume, length, thickness, and/or colors to eyelashes. However, mascara compositions that derive their performance primarily from waxes tend to become less resistant to oil and/or sebum, causing smearing, flaking, and/or color transferring after wearing for a certain amount of time. Furthermore, while conventional wax-based mascara can be used to assist in moderate curl formation eyelashes, users of mascara typically are forced to rely on particular applicators or eye-lash curling devices to enhance curl to any substantial degree.

WO 2010014328 describes the use of polystyrene sulfonate as a contractile polymer to apply to eyelashes.

WO2017044546 describes a film forming composition comprises a first non-crosslinking polyamide/polyacrylate copolymer and a second non-crosslinking polyamide copolymer comprising at least one amide; at least one quaternary ammonium containing monomer; and monomers having at least one amine functional group.

There remains a need for improved cosmetic compositions for application to keratinous materials such as mascaras having improved application properties, as well as ways in which to apply those compositions to provide maximum benefit.

Accordingly, one aspect of the present invention is a makeup kit for keratinous material which includes a cosmetic composition having good cosmetic properties such as, for example, curling properties, where the kit includes an applicator which allows easy application of the cosmetic composition having good cosmetic properties to keratinous material.

### SUMMARY OF THE INVENTION

The present invention relates to a kit for applying a cosmetic composition to hair, eyebrows and/or eyelashes comprising a cosmetic applicator and a cosmetic composition. The cosmetic applicator comprises a brush comprising a core having an outer surface and a longitudinal axis; a plurality of bristles protruding from the core and spaced apart along the longitudinal axis; and a high density zone that covers at least a portion of the outer surface of the core, the high density zone having a surface bristle density of up to 7 bristles per square millimeter of area of the outer surface of the core, and wherein the bristles have a length from about 0.6 mm to about 4 mm. The cosmetic composition has a viscosity of less than about 250 Pa·S when measured at a shear rate at 5 s⁻¹. Preferably, the cosmetic composition is a mascara.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

The accompanying drawings, which are incorporated in and constitute a part of the specification, illustrate one or more embodiments and, together with the description, explain these embodiments. The accompanying drawings have not necessarily been drawn to scale. Any values dimensions illustrated in the accompanying graphs and figures are for illustration purposes only and may or may not represent actual or preferred values or dimensions. Where applicable, some or all features may not be illustrated to assist in the description of underlying features. In the drawings:

FIG. 1 is a perspective view of a representative system according to the present disclosure, including a representative example of brush and a representative example of the container.

FIG. 2 is a perspective view of the brush of FIG. 1.

FIG. 3 is a partial side view of the brush of FIG. 2.

FIG. 4 is a section view of the brush of FIG. 2.

FIG. 5 is another partial side view of the brush of FIG. 2.

FIG. 6 is a perspective view of another embodiment of a brush according to one or more aspects of the present disclosure, the brush being suitable for use with the container of FIG. 1.

FIG. 7 is a partial side view of the brush of FIG. 6.

FIG. 8 is a front view of the brush of FIG. 6.

FIG. 9 is another partial side view of the brush of FIG. 6.

FIG. 10 is a perspective view of another embodiment of a brush according to one or more aspects of the present disclosure, the brush being suitable for use with a container such as is shown in FIG. 1.

FIG. 11 is a partial side view of the brush of FIG. 10.

FIG. 12 is a section view of the brush of FIG. 10.

FIG. 13 is another partial side view of the brush of FIG. 10.

FIG. 14 illustrates a representative method of using the brush of FIG. 10.

### DETAILED DESCRIPTION OF THE INVENTION

In the following description of the invention and the claims appended hereto, it is to be understood that the terms used have their ordinary and accustomed meanings in the art, unless otherwise specified.

“About” as used herein means within 5% of the indicated number (e.g. “about 10%” means 9.5%-10.5% and “about 2%” means 1.9%-2.1%).

“A” or “an” as used herein means “at least one.”

As used herein, all ranges provided are meant to include every specific range within, and combination of subranges between, the given ranges. Thus, a range from 1-5, includes specifically 1, 2, 3, 4 and 5, as well as subranges such as and 2-5, 3-5, 2-3, 2-4, 1-4, etc.

“Film former”, “film-forming polymer” or “film forming agent” or “co-film former” as used herein means a polymer or resin that leaves a film on the substrate to which it is applied, for example, after a solvent accompanying the film former has evaporated, absorbed into and/or dissipated on the substrate. For example, to evaluate whether a compound is a film former or film forming agent, a drawdown test may be performed by putting 5 to 10 grams of material on the center of a Leneta card stock (Black and White Opacity card Chart 2812 available from BYK Additives and Instruments of Geretsried, Germany) and using a 3 mil Drawdown Birdbar (also from Byk), spreading the material for across the sheet (8 in by 3 in) and allowing it to dry overnight. If the material forms a conformal coating and/or can picked up or scraped off with a razorblade to be removed as a free standing film, then it is film forming. Regardless, if it does not coat the card, cannot in any reasonable way be removed as a free-standing film and/or forms a loose powdery coating that rubs off readily onto one's finger, then it is not a film former.

“Wax” as used herein is a lipophilic fatty compound that is solid at ambient temperature (25° C.) and changes from the solid to the liquid state reversibly, having a melting temperature of more than 30° C. and, for example, more than 45° C., which can be as high as 150° C., a hardness of more than 0.5 MPa at ambient temperature, and an anisotropic crystalline organization in the solid state.

“Free of” or “devoid of” as used herein in connection with a particular element or ingredient means that the composition does not contain any of the element or ingredient. Thus, for example, “free of oils” or “devoid of oils” means that oils are omitted from the composition (that is, 0% by weight of the composition). “Essentially free of” or “Essentially devoid of” means that the composition can contain up to 0.2% by weight of the composition of the identified element or ingredient (for example, oils). “Substantially free of” or “Substantially devoid of” means that the composition can contain up to 0.33% by weight of the composition of the identified element or ingredient (for example, oils).

“Makeup Result” as used herein, refers to compositions where color remains the same or substantially the same as at the time of application, as viewed by the naked eye, after an extended period of time. “Makeup Result” may be evaluated by evaluating long wear properties by any method known in the art for evaluating such properties. For example, long wear may be evaluated by a test involving the application of a composition to keratin materials such as eyelashes and evaluating the color of the composition after an extended period of time. For example, the color of a composition may be evaluated immediately following application to keratin materials such as eyelashes and these characteristics may then be re-evaluated and compared after a certain amount of time. Further, these characteristics may be evaluated with respect to other compositions, such as commercially available compositions.

“Making up” as used herein means to provide decoration (for example, color) to keratin materials such as the eyelashes.

“Protecting” as used herein means to inhibit damage to keratin materials such as the eyelashes by providing a protective layer on the keratin materials.

“Substituted” as used herein, means comprising at least one substituent. Non-limiting examples of substituents for substitution include atoms, such as oxygen atoms and nitrogen atoms, as well as functional groups, such as hydroxyl groups, ether groups, alkoxy groups, acyloxyalkyl groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Water resistance” as used herein, means resistance of a material (substance) to the penetration of water, which may cause degradation of that material. The method implemented if assessment of this invention is further disclosed.

“Transfer resistance” as used herein refers to the quality exhibited by compositions that are not readily removed by contact with another material, such as, for example, a glass, an item of clothing or the skin, for example, when eating or drinking. Transfer resistance may be evaluated by any method known in the art for evaluating such. For example, transfer resistance of a composition may be evaluated by a “kiss” test. The “kiss” test may involve application of the composition to human keratin material such as hair, skin or lips followed by rubbing a material, for example, a sheet of paper, against the hair, skin or lips after expiration of a certain amount of time following application, such as 2 minutes after application. Similarly, transfer resistance of a composition may be evaluated by the amount of product transferred from a wearer to any other substrate, such as transfer from the hair, skin or lips of an individual to a collar when putting on clothing after the expiration of a certain amount of time following application of the composition to the hair, skin or lips. The amount of composition transferred to the substrate (e.g., collar, or paper) may then be evaluated and compared. For example, a composition may be transfer resistant if a majority of the product is left on the wearer's hair, skin or lips. Further, the amount transferred may be compared with that transferred by other compositions, such as commercially available compositions. In a preferred embodiment of the present invention, little or no composition is transferred to the substrate from the hair, skin or lips.

The compositions and methods of the present invention can comprise, consist of, or consist essentially of the essential elements and limitations of the invention described herein, as well as any additional or optional ingredients, components, or limitations described herein or otherwise useful.

Referred to herein are trade names for materials including, but not limited to polymers and optional components. The inventors herein do not intend to be limited by materials described and referenced by a certain trade name. Equivalent materials (e.g., those obtained from a different source under a different name or catalog (reference) number) to those referenced by trade name may be substituted and utilized in the methods described and claimed herein.

All percentages and ratios are calculated by weight unless otherwise indicated. All percentages are calculated based on the total weight of a composition unless otherwise indicated. All component or composition levels are in reference to the active level of that component or composition, and are exclusive of impurities, for example, residual solvents or by-products, which may be present in commercially available sources.

The following disclosure is to aid the reader in understanding the present invention, but it is not intended to vary or otherwise limit the meaning of the invention or terms/phrases describing the invention.

Kit for Applying a Cosmetic Composition to Keratinous Material

According to the present invention, a kit for applying a cosmetic composition to hair, eyebrows and/or eyelashes comprising a cosmetic applicator and a cosmetic composition is provided. Preferably, the cosmetic composition is a mascara.

Cosmetic Applicator

According to the present invention, a cosmetic applicator comprising a brush comprising a core having an outer surface and a longitudinal axis; a plurality of bristles protruding from the core and spaced apart along the longitudinal axis; and a high density zone that covers at least a portion of the outer surface of the core, the high density zone having a surface bristle density of up to 7 bristles per square millimeter of area of the outer surface of the core, and wherein the bristles have a length from about 0.6 mm to about 4 mm is provided for the kits of the present invention.

Exemplary details of the cosmetic applicator of the invention systems will now be described.

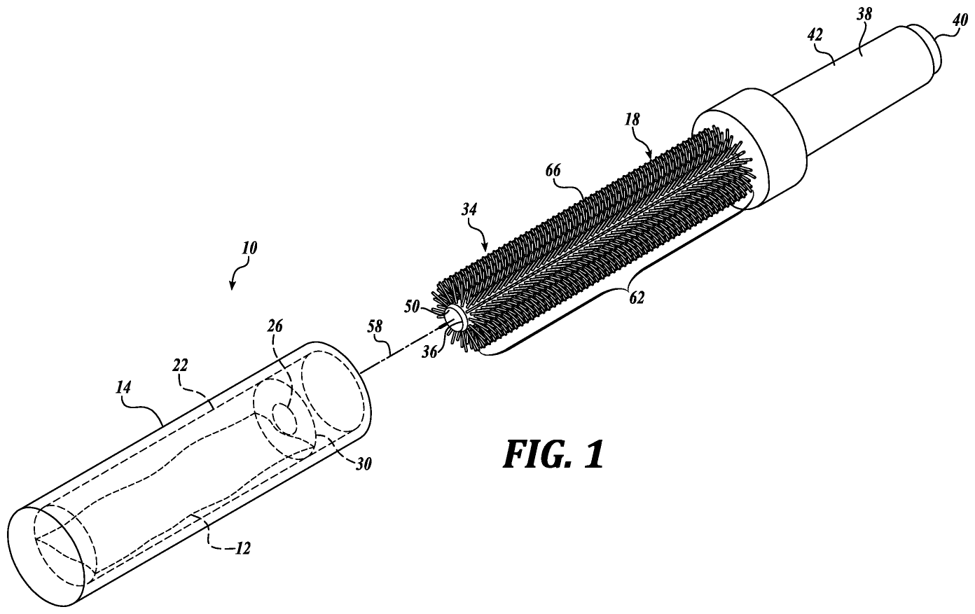

Referring now to FIG. 1, a representative system 10 is shown for storing and applying a formulation 12. In the embodiment shown, the system 10 includes a container 14 and a brush 18 having a plurality of bristles. The container 14 includes an internal chamber 22 for storing a formulation, e.g., mascara. A rigid or semi-rigid wiper 26 is affixed within the internal chamber 22, or formed integrally with the internal chamber 22 of the container 14, and is configured to “wipe” excess formulation 12 off the bristles of the brush 18 as the brush 18 is removed from the container 14. In the embodiment shown, the wiper 26 includes an internal opening 30 having a shape that may approximate a cross sectional shape of the brush 18. In some embodiments, such as embodiments having brushes with non-cylindrical cores, the wiper may have an internal opening that is not circular in order to approximate the cross section shape of the core. In some embodiments, the opening 30 may be somewhat smaller than the cross sectional shape of the brush 18. The diameter of the internal opening of the wiper may vary between embodiments, but generally is less than about 4.5 mm, such as between about 2 mm and about 4.5 mm for a wiper formed from a flexible material or between about 3 mm and about 4.5 mm for a wiper formed from a rigid material such as polyethylene.

The brush 18 may be releasably securable to the container 14, for example via a threaded coupling or other closure structure (not shown). The brush 18 is generally elongated, and includes a bristle section 34 and a handle 38. When the brush 18 is secured to the container 14, the bristle section 34 is inserted into the internal chamber 22 of the container 14 such that a distal end 36 extends through the internal opening 30 of the wiper 26 such that the bristle section 34 may contact the formulation 12 stored within the internal chamber 22. Once the bristle section 34 is removed, formulation 12 that has adhered to the brush 18 may then be applied to an object, such as hairs, by stroking the formulation-laden brush 18 against the object. The subject may occasionally reload the brush 18 by again inserting the distal end 36 into the internal chamber 22, optionally rotating the brush 18 within the chamber and/or shaking the container 14 to distribute formulation 12 around the brush 18, and then removing the brush 18.

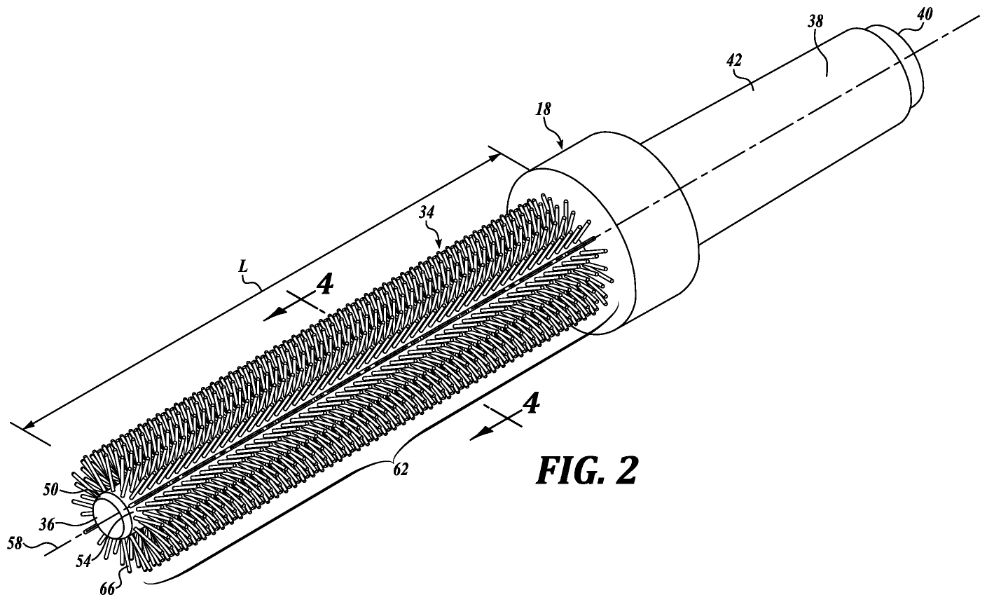

Referring now to FIG. 2, the brush 18 of FIG. 1 is shown without the container 14. It is contemplated that any of the brushes described herein may be sold as part of a system (such as system 10) that also includes a container having a wiper 26 and a formulation 12 contained within the internal chamber 22, or may be distributed or sold separately from the container.

As shown in FIG. 2, the bristle section 34 of the brush 18 includes a core 50 having a radial outer surface 54 and a plurality of bristles 66 projecting outwardly therefrom. The bristle section 34 of brush 18 extends longitudinally from the distal end 36 a distance L towards proximal end 40. The outer surface 54 of the core 50 has a surface area that corresponds directly with the volume of formulation that the brush 18 can hold. In particular, and with reference to FIG. 4, a formulation layer 74 may tend to form around the core 50. Such a formulation layer 74 advantageously enables transfer of the formulation to the hairs of a subject. That is, the larger the surface area of the core 50, the more formulation the brush 18 can hold, all else equal. The ability to hold more formulation may be advantageous. In addition to influencing how much formulation can be stored on the brush 18, the surface area of the core 50 is also a key factor in bristle density. As will be discussed in more detail below, the bristle density of a given brush can greatly affect its ability to efficiently and uniformly transfer formulation to hairs of a subject. Namely, when bristle density is too high relative to the surface area of the core, fine hairs may not be able to enter gaps between adjacent bristles, and formulation may tend to clump together.

Returning to FIG. 2, a longitudinal axis 58 is shown extending parallel to the core 50 through its center in order to facilitate visualization of the various features of the brush 18. The length of the core may vary between embodiments, but generally is about 10 mm to about 50 mm. In the embodiment of FIG. 2, the length L of core 50 is 30 mm. In other embodiments, the core may have a length L of 20 mm, 25 mm, 40 mm, or other length, although these core lengths are merely exemplary.

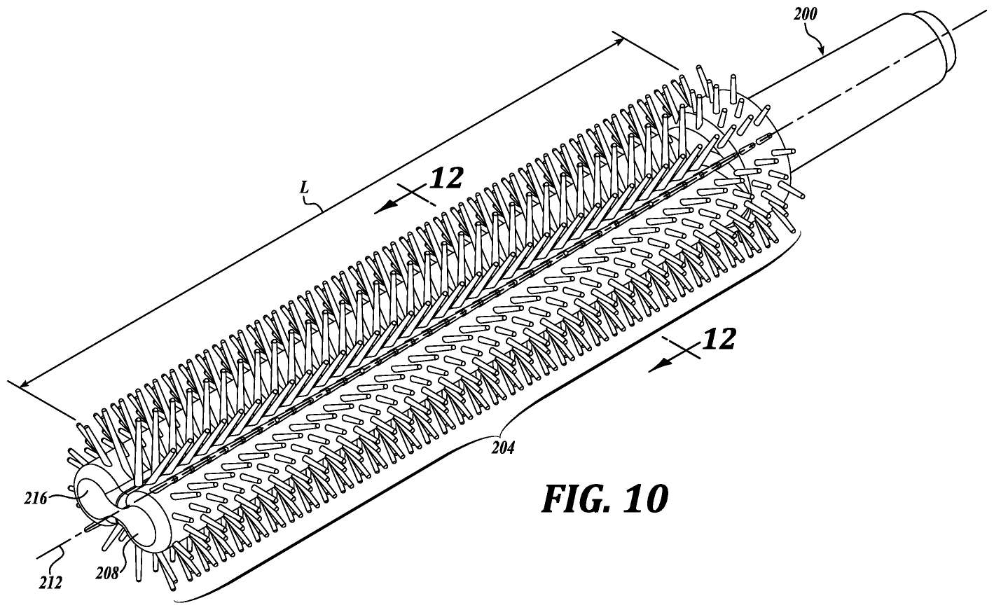

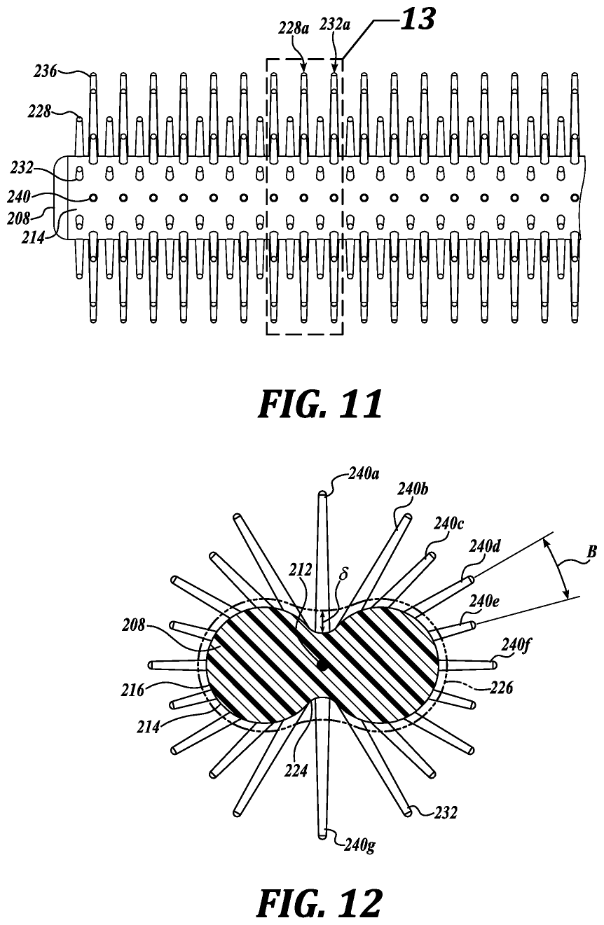

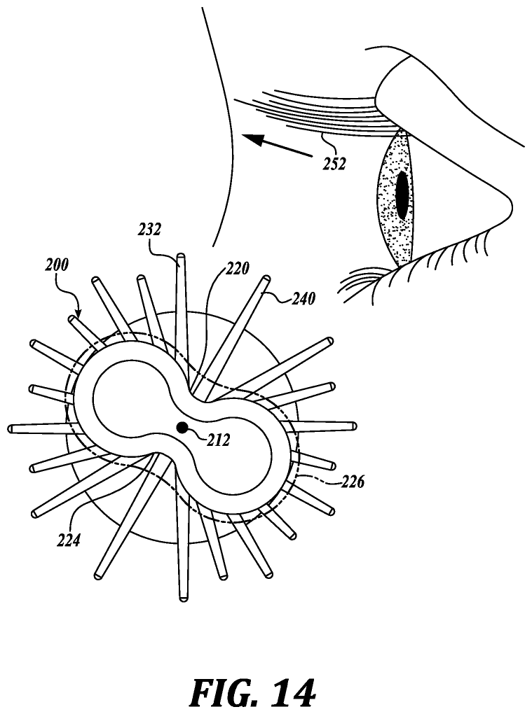

Generally, the core 50 has a cross sectional shape when viewed in a two-dimensional plane that is normal to the longitudinal axis 58. In some embodiments, the cross sectional shape is constant along the longitudinal axis. For example, referring to FIG. 4, the brush 18 has a cylindrical core 50 with a circular cross sectional shape when viewed in a plane that is normal to any point along the longitudinal axis 58. In some embodiments, the core 50 has an outside diameter D of 2.6 mm, which equates to a circumference of 2.6π mm. In embodiments where the bristled section of the core 50 has a length L of 30 mm, the total nominal bristled surface area is approximately 245 mm², not considering the surface area occupied by the bristles themselves. In other embodiments, the cross section shape is not constant along the longitudinal axis 58, but instead varies along the length L of core, or sections thereof. In yet other embodiments, the cross sectional shape remains constant along the longitudinal axis 58 but can vary in cross sectional area along the length L of core, or sections thereof. One example of such an embodiment is discussed below with respect to FIG. 10-14. The core may be formed from a non-metallic material such as a plastic material. Materials suitable for forming the core include thermoplastic materials including thermoplastic elastomers as well as thermoset materials. In other embodiments the core is essentially free of metallic materials such as those formed into wires and the like.

Brushes of the present disclosure include one or more high density zones having a plurality of bristles 66 that protrude radially outward from the core 50. The bristles conform to certain bristle density criteria that enable efficient and uniform transfer of formulations to fine hairs, such as eyelashes. The brush may include a single high density zone or a plurality of high density zones. In some embodiments, one or more high density zones may substantially make up the entire brush. For example, the brush 18 of FIGS. 1-5 includes a single high density zone 62 that has a length equal to the length L of the bristle section 34. The following discussion concerns such high density zones. Generally, the high density zone may include between about 500 to about 1,500 total bristles, for example about 600 to about 1,000 total bristles, or between about 600 and about 799 total bristles. It is contemplated that brushes of the present disclosure may have one or more zones that are not high density zones as described herein in addition to at least one high density zone.

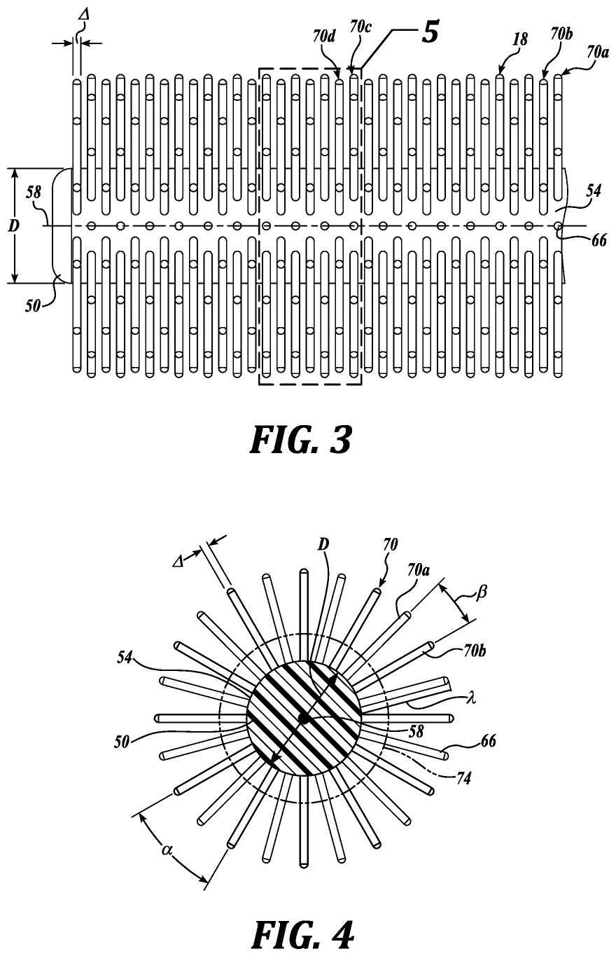

The bristles serve several important functions, for example storing formulation, breaking up formulation into smaller amounts, separating hairs of the subject, and transferring the formulation to the hairs of the subject. In the brush 18 of FIGS. 2-4, the high density 62 zone includes bristles 66 arranged in a plurality of bristle rings 70 that are spaced apart along the core 50, with each successive bristle ring 70_{a, c} staggered relative to each adjacent bristle ring 70_{b, d} about the longitudinal axis by an angle β of 15 degrees. In some embodiments, the brush 18 includes 100 bristle rings 70, although different embodiments may include a different number of rings. When viewed from both the side as in FIG. 3 and from the end as in FIG. 4, it can be seen that each bristle ring 70 is oriented substantially normally to the longitudinal axis 58 of the core 50. In other embodiments however, bristle rings may have one or more orientations that are not perpendicular to the longitudinal axis of the core. For example, a bristle ring may have an oblique orientation relative to the longitudinal axis, and may intersect with other bristle rings, subject to the limits of bristle density discussed below. In still other alternative embodiments, the bristles may not form discrete rings, but rather one or more continuous helixes around the core for example. As noted above with respect to angle β, bristle rings may have different angular orientations with respect to the longitudinal axis when viewed in a two-dimensional plane that is normal to the longitudinal axis. For example, in other embodiments, bristle rings may be offset from each other about the longitudinal axis by about 0 to about 90 degrees, for example about 5 degrees, about 10 degrees, about 20 degrees, about 22.5 degrees, about 25 degrees, or another angle, etc.

Each bristle ring 70 typically, but not always, extends all the way around the core 50. Referring to FIGS. 2-4, each bristle ring 70 extends entirely around the outer surface 54 of the core 50, i.e., 360 degrees around the longitudinal axis 58. These “full” bristle rings 70 are preferred for applying formulation to fine hairs. However, it is contemplated that in other embodiments, a high density zone may include one or more bristle rings that only partially extend around the outer surface of the core, i.e., partially around the longitudinal axis, for example about 90 degrees, about 120 degrees, about 180 degrees, or another value that is less than 360 degrees.

Each bristle may generally be formed from. any thermoplastic material that is optionally relatively rigid, e.g.: styrene-ethylene-butylene-styrene (SEBS); a silicone rubber; latex rubber; a material having good slip; butyl rubber; ethylene-propylene terpolymer rubber (EPDM); a nitrile rubber; a thermoplastic elastomer; a polyester, polyamide polyethylene, or vinyl elastomer; a polyolefin such as polyethylene (PE) or polypropylene (PP); polyvinyl chloride (PVC); ethyl vinyl acetate (EVA); polystyrene (PS); SEBS; styrene-isoprene-styrene (SIS); polyethylene terephthalate (PET); polyoxymethylene (POM): polyurethane (PU); styrene acrylonitrile (SAN); polyamide (PA); or polymethyl methacrylate (PMMA). It is also possible to use a ceramic, e.g. an alumina-based ceramic, a resin, e.g. a urea formaldehyde type resin, possibly a material filled with graphite. In particular, it is possible to use materials known under the trade names Teflon, Hytrel®, Cariflex®, Alixin®, Santoprene®, Pebax®, Pollobes®, this list not being limiting. Preferably, each bristle is formed from at least one thermoplastic elastomer.

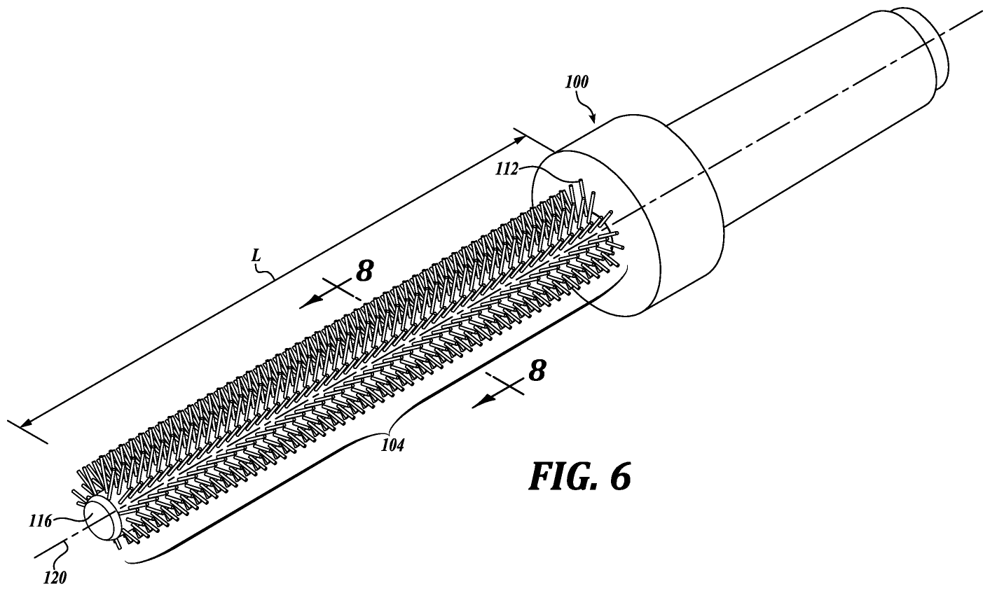

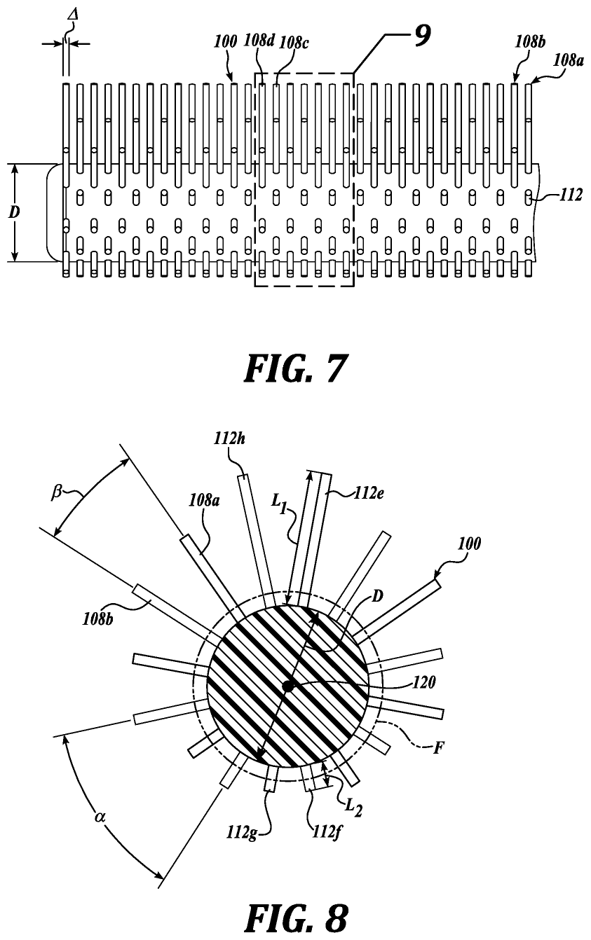

The dimensions of individual bristles may vary between embodiments. In particular, the bristle length and bristle diameter can greatly influence brush performance. As used herein, bristle length is measured as the exposed length of a bristle that projects radially outwardly beyond the outer surface 54 of the core 50—not the length considering any additional bristle length below the outer surface of the core. It has been discovered that in high density zones, bristle lengths of about 0.6 mm to about 4.0 mm are preferred for applying formulations to fine hairs, for example bristle lengths of about 0.6 mm, about 1.0 mm, about 1.25 mm, about 1.5 mm, about 2.0 mm, about 3.0 mm, and about 3.5 mm. Referring to FIG. 4, each bristle 66 of the brush 18 has a length A, of 2.0 mm, which reflects the length of each bristle 66 that extends beyond the outer surface 54 of the core 50. The range of appropriate bristle lengths for a given application may depend on the bristle material. For example, bristles formed from thermoplastic elastomers may have lengths ranging from about 0.6 mm to about 4.0 mm. Further, a single brush, and even a single bristle ring, may include bristles of more than one length. The lengths of successive bristles may vary, for example in a continuously increasing or decreasing pattern, an alternating pattern, or another pattern, such that the different bristle lengths provide targeted advantages. In some embodiments, no more than, for example, 8, 7, 6, 5, 4, 3, or 2 bristles may have the same bristle length. In some embodiments, a single bristle ring may include one or more bristles with a first bristle length and one or more bristles with a second bristle length, which may differ by about 0.1 mm to about 3.5 mm, e.g., about 1.0 mm, about 2.0 mm, or about 3.0 mm. In some embodiments, for example, 1, 2, 3, 4, 5, or more consecutive bristles within the same bristle ring of the high density zone may have the same bristle length. In some embodiments, no two consecutive bristle rings may include bristles of the same bristle length. These features may advantageously provide bristles best suited for different fine hair diameters on a single brush (and even within a single high density zone). Such examples are discussed below with respect to the brushes of FIGS. 6-14.

Bristle diameter, measured where the bristle meets the outer surface of the core, should generally be about 0.05 mm to about 0.35 mm, e.g., about 0.1 mm, about 0.125 mm, about 0.15 mm, about 0.175 mm, and about 0.2 mm, subject to the bristle density limits discussed below. Bristles having diameters in this range generally exhibit sufficient stiffness while also permitting the brush to have bristle density within the limits discussed below. For example, the brush 18 of FIGS. 2-4 has bristles with a diameter Δ, of about 0.175 mm.

The number of bristles per bristle ring may vary between embodiments. “Full” bristle rings, i.e., bristle rings that extend completely around the outer surface of the core (i.e., 360 degrees about the longitudinal axis), may each include 2 to 30 bristles in high density zones, and preferably 7 to 15 bristles per ring, for example 7, 8, 9, 10, 11, 12, 13, 14, 15, 16, 18, 20, 22, 24, 26, 28, 30, or any other number of bristles in that range. In full rings, the number of bristles is the “full ring bristle count.” For example, each bristle ring 70 of the brush 18 of FIGS. 2-5 includes 12 bristles, which are spaced apart about the longitudinal axis 58 by an angle α of about 30 degrees. Therefore, each bristle ring 70 has a full ring bristle count of 12 bristles.

In other embodiments, a partial bristle ring, i.e., a ring sector that does not extend completely around the outer surface of the core (i.e., that does not extend 360 degrees about the longitudinal axis of the core), may also include 2 to 30 bristles. For example, a partial bristle ring may include a sector that extends only 180 degrees about the longitudinal axis and includes 6 bristles in that 180 degree sector, each bristle being spaced apart from the adjacent bristles by an angle α of 30 degrees. Similarly, a single bristle ring may include bristles that have different angular spacing a about longitudinal axis of the core. For example, a single bristle ring may include a first 120 degree sector with 3 bristles spaced apart by 40 degrees, a second 120 degree first sector with 4 bristles spaced apart by 30 degrees, and a third 120 degree sector with 5 bristles spaced apart by 24 degrees. These configurations are merely exemplary. Other embodiments may include partial or full bristle rings having a different number of bristles and different angular spacing, within the limits of bristle density discussed below.

In embodiments with partial bristle rings or bristle rings with heterogeneous angular spacing, it can be useful to think of such partial or heterogeneous bristle rings by reference to an equivalent “full-ring bristle count,” which may be calculated by multiplying a) the number of bristles in the densest angular sector of the partial ring and b) the number of such angular sectors that would fit within a 360 degree ring. For example, in the first example from the previous paragraph, the partial bristle ring that extends 180 degrees around the core and includes 6 bristles would have a full-ring bristle count of 6 bristles*(360/180)=12 bristles. In the second example from the previous paragraph, the 3-sector heterogeneous bristle ring has a full-ring bristle count based upon its densest sector, i.e., 5 bristles*(360/120)=15 bristles.

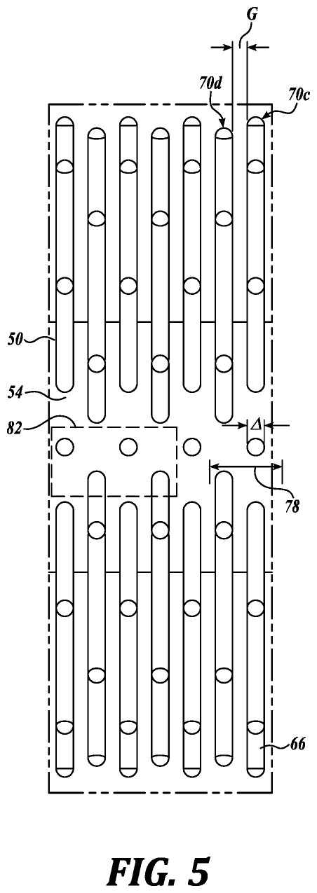

The spacing between adjacent bristle rings is another important variable within high density zones. As noted above, fine hairs generally have diameters ranging from about 0.05 mm to about 0.1 mm. Adjacent bristle rings should be sufficiently spaced apart along the longitudinal axis such that fine hairs may enter that space—generally at least 0.1 mm. Insufficient spacing (e.g., less than 0.1 mm) not only makes it difficult for individual hairs to enter the spacing between bristles, but may also lead to undesirable clumping because the formulation does not have space to break apart. On the other hand, excessive spacing between adjacent bristles may result in inadequate transfer of formulation to the hairs of a subject because individual hairs pass between bristles without making contact with formulation stored on and around the bristles. This condition leads to inefficient formula transfer. Excessive spacing may also result in inadequate separation of the hairs, which can lead to irregular clumping of formulation on the hairs. To overcome these challenges, adjacent bristle rings of the inventive brushes disclosed herein may be spaced apart by a gap of between about 0.1 mm and about 0.3 mm, subject to the bristle density limitations discussed below. The aforementioned gap refers to the distance, measured along the longitudinal axis, between the nearest surfaces of adjacent bristle rings when viewed in a two-dimensional plane parallel to the longitudinal axis, and is not affected by an axial offset between adjacent bristle rings. For example, referring to FIG. 5, adjacent bristle rings 70_{c}, 70_{d} are spaced apart by a gap G of about 0.15 mm.

Bristle density is a key variable in high density zones configured to efficiently and uniformly transfer formulations—especially gummy formulations—to fine hairs. More than one measure of bristle density impacts brush performance. One key measure of bristle density is the number of bristles relative to the core length, i.e., “linear bristle density.” It has been discovered that in order to optimally transfer gummy formulations to fine hairs, in certain embodiments, a high density zone should have a linear bristle density of 13 to 31 whole bristles per 0.5 mm of length along the outer surface of the core measured parallel to the longitudinal axis. For example, linear bristle densities of 14, 15, 16, 17, 18, 19, 20, 21, 22, 23, 24, 25, 26, 27, 28, 29, 30, and 31 whole bristles per 0.5 mm of core length may be suitable. In the inventive brushes disclosed herein, 1 to 3 “full” bristle rings will fit within each 0.5 mm of core length within the high density zone, measured along the longitudinal axis. As a metric, the linear bristle density captures several specifications that impact brush performance, including bristle diameter (Δ), angular bristle spacing within a bristle ring (α), as well as spacing between bristles of adjacent bristle rings (G). When linear bristle density exceeds 31 bristles per 0.5 mm of core length, the bristles tend to not allow fine hairs to enter the gaps between bristles and tend to clump the formulation. The “full-ring linear bristle density” is calculated by first converting all bristle rings within the high density zone to their “full-ring bristle count,” then measuring a 0.5 mm length parallel to the longitudinal axis of the core, and counting the number of “full-ring” bristles within the high density zone that would be encompassed by the 0.5 mm length. For example, referring again to FIGS. 2-5, the high density zone 62 of the brush 18 has 100 full bristle rings 70, each with 12 bristles (and therefore each bristle ring 70 has a “full ring” bristle count of 12 bristles). Each bristle 66 has a 0.175 mm diameter, and adjacent bristle rings 70 are spaced apart by gap G of 0.15 mm. Thus, two full bristle rings 70_{c},70_{d} “fit” within a 0.5 mm length 78 measured along the core 50 (e.g., 0.175 mm+0.15 mm+0.175 mm=0.5 mm). Because each bristle ring 70 has a full ring bristle count of 12 bristles, the high density zone 62 has a full ring linear bristle density of 12 bristles*2 rings=24 bristles per 0.5 mm length along the core 50. It is important to note that other brushes having different bristle counts, bristle diameters, gaps, and angular spacing could achieve the same 24 bristle full-ring bristle density per 0.5 mm of core length. For example, a hypothetical brush with 8 bristles per full ring, each bristle having a 0.1 mm diameter, and adjacent rings spaced apart by 0.1 mm, would also have a full ring bristle density of 24 bristles per 0.5 mm of core length because three full bristle rings would fit within a 0.5 mm core length.

Another key measure of bristle density is the number of bristles relative to the core surface area, or “surface bristle density.” It has been discovered that in order to efficiently and uniformly transfer formulation to fine hairs, a high density zone should have a surface bristle density of up to 7 bristles per square millimeter of surface area of the core (i.e., the nominal core surface area, not considering the surface area occupied by the bristles themselves), preferably 2-7 bristles per square millimeter of surface area of the core, and preferably 4-6 bristles per square millimeter of surface area of the core, as brushes with a surface bristle density that exceeds 7 whole bristles per square millimeter of surface area (i.e., 8/mm² or greater) tend to not allow fine hairs to enter the gaps between bristles and tend to clump the formulation. As a metric, the surface bristle density captures several specifications that influence brush performance, including bristle diameter (Δ), angular bristle spacing (α), spacing between bristles of adjacent bristle rings along the longitudinal axis (G), and the amount of core surface area that is available to store formulation. The surface bristle density of a high density zone is the greater of a local measurement and an average measurement—neither should exceed 7 bristles per square millimeter of surface area. To determine the local surface bristle density within a high density zone, a 1 mm by 1 mm square in a plane that is tangential to the surface of the core is drawn, and then the number of whole bristles that fit within that 1 mm×1 mm square is counted. For example, referring to the detail view of FIG. 5, 4 whole bristles fit within the 1 mm×1 mm box 82 that is tangential to core 50, i.e., a local surface bristle density of 4 whole bristles/mm². By comparison with the local surface bristle density, the average surface bristle density is determined by dividing the total number of bristles covering the core surface area corresponding to the high density zone, by the radial outer surface area of the high density zone itself. Referring again to FIGS. 2-5, the brush 18 has 1,200 bristles within the high density zone 62 (100 bristle rings, each with 12 bristles), and the cylindrical core 50 has a 2.6 mm outer diameter D, and a 30 mm length L, which equates to a 245 mm² surface area (2.6π mm×30 mm). Therefore, the brush 18 has an average surface bristle density of 1,200 bristles/245 mm²=4.9 bristles/mm² (i.e., 4 whole bristles). From this, it is evident that the local and average surface bristle densities are the same: 4 whole bristles per square millimeter.

To clarify, according to certain embodiments, brushes useful according to the present disclosure may have (1) a high density zone with a linear surface bristle density 13 to 31 whole bristles per 0.5 mm of length along the outer surface of the core measured parallel to the longitudinal axis and (2) a surface bristle density of up to 7 bristles per square millimeter of core surface area (taken as the greater of the local or average surface bristle density measurements described above).

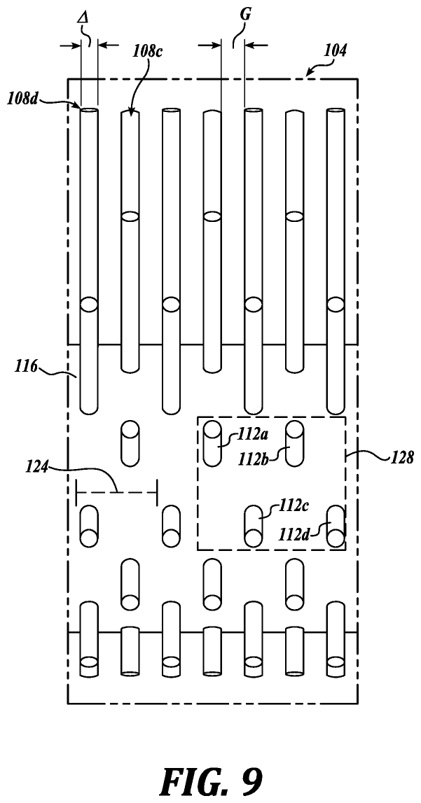

Referring now to FIGS. 6-9, another non-limiting example of a brush 100 is shown having a single high density zone 104 that includes 100 bristle rings 108, each having 8 bristles with a 0.15 mm diameter Δ. Whereas each bristle ring of the brush of FIGS. 1-5 has 12 bristles spaced apart by an angle α of 30 degrees, each bristle ring 108 of the brush 100 of FIGS. 6-9 has 8 bristles 112 spaced evenly about the longitudinal axis 120 at an angle α of 45 degrees. In other words, each bristle ring 108 as a full ring bristle count of 8. Adjacent bristle rings 108ₐ, 108_{b} are offset about the longitudinal axis 120 by angle β, which is 22.5 degrees. Adjacent bristle rings 108 are evenly spaced along the longitudinal axis by a gap G of 0.2 mm along a cylindrical core 116 having a length L of 25 mm, a diameter D of 2.5 mm, the core 116 having a constant cross sectional shape and dimensions along a longitudinal axis 120. In this embodiment, the high density zone extends the entire length of the core 116, and therefore has the same length. As shown in FIG. 9, two full bristle rings 108_{c, d} fit within a 0.5 mm length 124 along the core; therefore, the brush has a linear bristle density of 16 bristles per 0.5 mm of core length. FIG. 9 also shows that the brush 100 has a local surface bristle density of 4 bristles, since whole bristles 112ₐ, 112_{b}, 112_{c}, and 112_{d} fit within the 1 mm×1 mm area box 128. The average surface bristle density is approximately 4.1 bristles per mm² (i.e., 4 whole bristles), calculated as the total number of bristles 112 within the high density zone (100 rings×8 bristles per ring=800 bristles) divided by the surface area of the high density zone 104 (2.5π mm×25 mm=196.3 mm²). Thus, the high density zone 104 has a linear bristle density of 13 to 31 whole bristles per 0.5 mm of core length, and a surface bristle density of 3 to 5 whole bristles per mm².

The brush 100 of FIGS. 6-9 provides an additional advantage because each bristle ring 108 includes bristles 112 having different lengths. Referring to FIG. 8, when the brush 100 is viewed in a plane that is normal to the longitudinal axis 120, it can be seen that the bristle 112ₑ has a first length L₁, while the bristle 112_{f} has second length L₂. Moving clockwise from bristle 112ₑ to bristle 112_{f}, successive pairs of bristles 112 have shorter lengths than the preceding pairs of bristles 112. Similarly, moving clockwise from bristle 112_{g} to bristle 112ₕ, successive bristle pairs have a longer length than the preceding bristle pair. Advantageously, this aspect enables the brush 100 to efficiently and uniformly transfer formulation to a wide variety of fine hairs, thus making the brush 100 suitable for a greater number of potential subjects. The brush 100 of FIGS. 6-9 is a non-limiting example of this concept. Other brushes may include bristles having different lengths.

Brushes of the present disclosure may provide additional advantages by including at least one external recess for holding formulation. Such recesses are formed within, or by, the outer surface of the core, which recesses then hold formulation by surface tension. By storing formulation, the recesses reduce the frequency with which a brush must be reloaded with formulation, and also provides more formulation to transfer to the hairs of a subject in a single stroke. Such recesses may cooperate with other structure(s) designed to store formulation, e.g., cavities formed with the core of the brush, but are described herein as distinct from such “internal” cavities. The recesses may be formed by molding the core to a particular shape that inherently includes recesses, and/or by removing material from the core in a separate processing step. Cores having recesses may have organic or geometric cross-sectional shapes, which shapes and dimensions may be constant or may vary along a longitudinal axis. Such recesses may have a depth ranging from about 0.1 mm to about 1.5 mm, and may have a length ranging from about 1.0 mm to the entire length of the core.

Referring now to FIGS. 10-14, a brush 200 is shown having a high density zone 204 and embodies several advantages disclosed herein. Rather than a cylindrical core, the brush 200 includes a core 208 having an hourglass cross sectional shape. The hourglass shape is evident when the brush 200 is viewed along a longitudinal axis 212 as in FIG. 12. A spline 216 defines the hourglass shape of an outer surface 214 of the core 208. The spline 216 in an embodiment has a spline length of about 9.7 mm. The high density zone 204 extends along a length L of about 21.9 mm in some embodiments. Therefore, the outer surface area of the hourglass-shaped core 208 within the high density zone 204 is 9.7 mm×21.9 mm=212.8 mm² in this embodiment.

Referring still to FIG. 12, the core 208 advantageously includes a channel-shaped or groove-shaped first recess 220 and an identical second recess 224 located on an opposite side of the core 208. The recesses 220, 224 enable the brush 200 to hold more formulation, which is represented as layer 226 in FIG. 12. Each recess 220, 224 has a depth δ that is measured relative to a plane that is tangential to two radial-outermost points of the core 208. The depth δ of recess 220 is 0.8 mm, but in other embodiments may range from 0.1 mm-1.5 mm. The first and second recesses 220, 224 extend along the entire length L of the high density zone 204 or sections thereof. The depth δ and length L both correspond directly to a volume of the recesses 220, 224.

Many variations in the quantity, shape, and size of recesses are contemplated, and any brush of the present disclosure may include one or more such recesses—not just the embodiment of FIGS. 10-14. For example, in some embodiments, the core may have a tri-lobe cross-sectional shape that creates three recesses, a cloverleaf shape that creates four recesses, or a geometric shape that includes one or more recesses, such as a star shape. Other embodiments (not shown) may include only a single recess, or a greater number of recesses about the core, e.g., 5, 6, 7, 8, 9, 10, or more recesses. Whereas the recesses 220, 224 of FIGS. 10-14 form channels or grooves in the core 208, recesses in other embodiments may form divots, helixes, axially-spaced rings, and other shapes. In embodiments having a plurality of recesses, it is not essential that all recesses are identical; rather, the recesses may differ relative to each other in length, depth, shape, and other characteristics.

In use, the formulation layer 226 surrounds the core 208 and occupies the recesses 220, 224. As is evident from FIG. 12, the formulation layer 226 has greater depth in the location of the recesses 220, 224. This additional formulation stored around the core 208 enables the brush 200 to transfer more formulation to the hairs of a subject without reloading the brush 200.

The high density zone 204 of the brush 200 of FIGS. 10-14 includes sixty-seven bristle rings spaced apart by 0.1 mm to about 0.2 mm. The number of bristles per ring varies—each odd bristle ring 228 has 8 bristles (designated 232) and each even bristle ring 236 has 12 bristles (designated 240), for a total bristle count of 668 bristles. Each successive bristle ring 228, 236 is offset from each preceding and succeeding bristle ring 228, 236 by an angle β of about fifteen degrees, such that when viewed along the longitudinal axis 212 as in FIG. 12, 22 distinct bristles 232, 240 are visible. Each bristle 232, 240 has a base diameter of 0.18 mm.

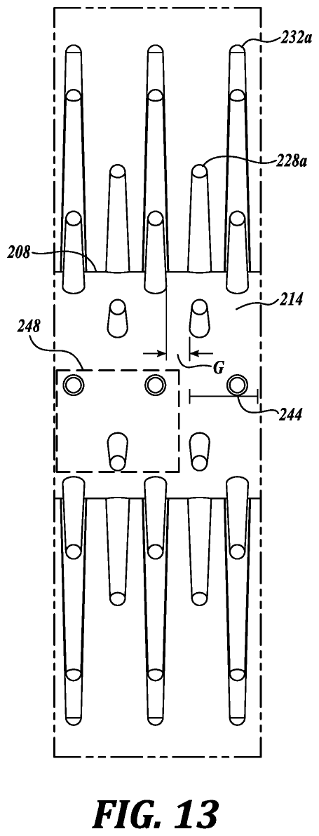

The bristle density of the brush 200 falls within the parameters outlined above. As shown in FIG. 13, two bristle rings 228ₐ, 232ₐ fit within a 0.5 mm length 244 measured along the core 208. Given that alternating bristle rings 228, 236 have 8 and 12 bristles 232, 240, respectively, this equates to a linear bristle density of 20 bristles per 0.5 mm of core length. The local surface bristle density is 3 whole bristles per square millimeter of core surface area, as visualized by the 1 mm×1 mm box 248 in FIG. 13. The average surface bristle density is calculated by dividing 668 bristles by the 212.8 mm² surface are of the high density zone, or 3.1 bristles per square millimeter (3 whole bristles).

As yet another advantage, the hourglass-shaped core 208 advantageously causes the bristles 232, 240 to have a plurality of bristle lengths. Referring again to FIG. 12, it is evident that the bristles 240ₐ and 240_{g} have the longest bristle length, as they project radially outwardly from the lowest point in the first and second recesses, 220, 224, respectively. Moving clockwise from the bristle 240ₐ, the next visible bristle 240_{b} has a second bristle length, which is less than the first bristle length because the bristle 240_{b} does not extend from the lowest point in the first recess 220. Moving clockwise again, the bristle 240_{c} has a third bristle length, which is less than the first and second bristle lengths because it is a shorter bristle and also because it projects from a relatively higher point on the core 208. Likewise, the bristle 240_{d} has a fourth bristle length, the bristle 240ₑ has a fifth bristle length, and the bristle 240_{f} has a sixth bristle length. From this, it is apparent that shape of the core 208 causes bristles 232, 240 to have different bristle lengths.

In use, a subject may use any of the brushes described herein to apply formulation to hair, such as eyelashes. With reference to the brush 200 of FIGS. 10-14, a subject may first load the brush 200 with formulation by inserting one end into a formulation-storing container (such as is shown in FIG. 1), withdrawing the brush 200, and stroking the brush 200 against one or more hairs 252. Optionally, before stroking the brush 200 against the hairs 252, the subject may selectively rotate the brush 200 about its longitudinal axis 212 before stroking the brush 200 such that hairs of the subject will pass through bristles 232, 240 extending from either recess 220, 224 during a stroke. In other words, the subject may rotate the brush 200 to align the first or second recess 220, 224 with the hairs 252. This step may advantageously increase the amount of formulation that is transferred to the hairs during a subsequent stroke. Optionally, the subject may selectively rotate the brush 200 before stroking such that bristles 232, 240 having a particular bristle length (e.g., a first, second, third, fourth, fifth, or sixth bristle length) will contact the hairs. This step may advantageously position bristles 232, 240 that are best-suited for the hair type of a subject to make contact with the hairs 252 during a stroke. The subject may then perform one or more strokes (preferably outward strokes) with the brush 200 against the hairs 252 in order to transfer formulation to the hairs 252, performing any of the steps described above in between strokes. Optionally, the subject may rotate the brush 200 during a stroke or otherwise while the brush is in contact with the hairs 141, in order to separate the hairs 252 and/or increase the amount of formulation transferred to the hairs 252.

In summary, inventive brushes of the present disclosure are configured to efficiently and uniformly transfer formulations, especially gummy formulations, to fine hairs. Such brushes include at least one high density zone having a linear bristle density of 13 to 31 whole bristles per 0.5 mm of core length and a surface bristle density of 3 to 5 whole bristles per square millimeter of core surface area. This configuration enables fine hairs to enter gaps between bristles and also enables formulation to break apart between the bristles, contrary to known dense brushes. In addition, brushes may have more than one bristle length, which advantageously enables a single brush to efficiently and uniformly transfer formulation to different hair sizes. In addition, brushes may include one or more recesses formed on or in the core, which enable the brushes to store a greater amount of formulation, which advantageously reduces the frequency with which a brush must be reloaded with formulation, and also provides more formulation to transfer to the hairs of a subject in a single stroke.

The detailed description set forth above in connection with the appended drawings is intended as a description of exemplary embodiments of the disclosed subject matter and is not intended to represent the only embodiments of the cosmetic applicator. The exemplary embodiments described in this disclosure are provided merely as examples or illustrations of a cosmetic applicator and should not be construed as preferred or advantageous over other embodiments. The illustrative examples provided herein are not intended to be exhaustive or to limit the disclosure to the precise forms disclosed. Similarly, any features and/or process steps described herein may be interchangeable with other features and/or process steps, or combinations of features and/or process steps, in order to achieve the same or substantially similar result.

In the foregoing description, numerous specific details are set forth in order to provide a thorough understanding of the exemplary embodiment of the present disclosure. It will be apparent to one skilled in the art, however, that many embodiments of the present disclosure may be practiced without some or all of the specific details. In some instances, well-known features, subassemblies, and/or process steps have not been described in detail in order not to unnecessarily obscure various aspects of the present disclosure. Further, it will be appreciated that embodiments of the present disclosure may employ any combination of features described herein. For instance, any feature or configuration described above with respect to one wiping assembly may be adapted for use with any other wiping assembly.

Although certain descriptive terms have been used to illustrate or describe certain aspects or benefits of the present invention, they should not be seen as limiting. For instance, the present disclosure also includes references to directions, such as “distal,” “proximal,” “upward,” “downward,” “top,” “bottom,” “first,” “second,” etc. These references and other similar references in the present disclosure are only to assist in helping describe and understand the exemplary embodiments and are not intended to limit the claimed subject matter to these directions. The term “cosmetic formulation” or “cosmetic” should be interpreted broadly to include any cosmetic formulation, beauty product, lotion, lacquer, etc., generally applied to the skin, eyes, nails, or other body part of a person. Moreover, it should be appreciated that the cosmetic applicators may also be adapted for other non-cosmetic uses, such as applying medicine, paint, etc., to a desired body part or surface.

The present disclosure may also reference quantities and numbers. Unless specifically stated, such quantities and numbers are not to be considered restrictive, but exemplary of the possible quantities or numbers associated with the present disclosure. Also in this regard, the present disclosure may use the term “plurality” to reference a quantity or number. In this regard, the term “plurality” is meant to be any number that is more than one, for example, two, three, four, five, etc. The terms “substantially,” “about,” “approximately,” etc., mean plus or minus 5%. For the purposes of the present disclosure, the phrase “at least one of A, B, and C,” for example, means (A), (B), (C), (A and B), (A and C), (B and C), or (A, B, and C), including all further possible permutations when greater than three elements are listed.

The principles, representative embodiments, and modes of operation of the cosmetic applicator of the present disclosure have been described in the foregoing description. However, aspects of the present disclosure, which are intended to be protected, are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure as claimed.

Cosmetic Composition

According to the present invention, a cosmetic composition for application to hair, eyebrows and/or eyelashes is provided. Preferably, the cosmetic composition is a mascara.

According to preferred embodiments, the cosmetic composition of the present invention possesses a viscosity of less than about 250 Pa·S when measured at a shear rate at 5 s⁻¹, preferably 225 Pa·S or less, preferably 200 Pa·S or less and preferably 175 Pa·S or less, and preferably greater than 10 Pa·S, preferably greater than 20 Pa·S, and preferably greater than 25 Pa·S, including all ranges and subranges therebetween including, for example, 10 Pa·S to 250 Pa·S, 20 Pa·S to 200 Pa·S, 15 Pa·S to 150 Pa·S, 40 Pa·S to 100 Pa·S, etc. as measured using, for example, magnetic bearing rheometer such as the Discovery HR-3 rheometer from TA Instruments of New Castle, Del., available from TA Instruments of New Castle, Del.

In accordance with the present invention, the cosmetic composition can be in any form and can contain any ingredient typical of cosmetic compositions for application to hair, eyebrows and/or eyelashes.

The cosmetic compositions can be in any form such as, for example, an anhydrous composition, an oil-in-water (O/W) emulsion including a silicone-in-water emulsion, a water-in-oil (W/O) emulsion including a water-in-silicone emulsion, a multiple emulsion, etc.

According to preferred embodiments of the present invention, cosmetic compositions of the present invention comprise water and at least one film-forming polymer comprising at least one cyclic group selected from the group consisting of cyclic amides, cyclic amines, and mixtures thereof. Preferably, the at least one film-forming polymer comprising at least one cyclic group selected from the group consisting of cyclic amides, cyclic amines, and mixtures thereof is non-ionic and water-soluble or water-dispersible. Without wishing to be bound by theory, it is believed that the presence of a sufficient amount of the at least one film forming polymer comprising at least one cyclic group selected from the group consisting of cyclic amides, cyclic amines, and mixtures thereof in the compositions of the present invention results in a curling effect on hair, eyebrows and/or eyelashes after application to the hair, eyebrows and/or eyelashes (and after the applied composition has dried).

According to preferred embodiments, the film forming polymer comprising at least one cyclic group selected from the group consisting of cyclic amides, cyclic amines, and mixtures thereof has a weight average molecular weight in a range from about 10,000 daltons to about 1,000,000 daltons, preferably from about 20,000 daltons to about 800,000 daltons, preferably from about 50,000 daltons to about 600,000 daltons, and preferably from about 100,000 daltons to about 500,000 daltons, including all ranges and subranges therebetween such as, for example, 15,000 daltons to 900,000 daltons, 200,000 daltons to 400,000 daltons, 10,000 daltons to 150,000 daltons, etc.

Preferably, the cyclic amide group and/or cyclic amine group of the at least one film forming polymer comprise one or more aromatic or aliphatic ring structures. Preferably, the rings have a size of from 4 to 10 ring members, preferably 5 to 8 ring members, and preferably 5 to 6 ring members, including all ranges and subranges therebetween.

Preferably, the cyclic amide group and/or cyclic amine group of the at least one film forming polymer are polymerizable ethylenically unsaturated monomers having a cyclic amine residue or a cyclic amide residue. Accordingly, the cyclic amide groups or monomers of the film-forming polymers useful in the present invention may include cyclic amide residues that are, or include, heterocyclic ring structures such as lactams and the like such as, for example, α-Lactam, β-lectern, γ-lactam, δ-lactam, and ε-lactam. Preferably, the cyclic amide is a pyrrolidone (a γ-lactam) a caprolactam, or combinations thereof.

Preferred cyclic amine groups include various heterocyclic amines such as, for example, azoles, pyrroles, pyrrolidines, carbamates, and the like. Preferably, the cyclic amine group is an imidazole.

Optionally, the film forming polymer comprising at least one cyclic group selected from the group consisting of cyclic amides, cyclic amines, and mixtures thereof may further comprise other groups in addition to the cyclide amide groups and/or cyclic amine groups.

If present, the additional group(s) are preferably acrylamide monomer(s), preferably having one or more —C₃H₅NO functional groups. Specific examples of such additional groups include, but are not limited to, (meth)acrylamides.

An example of a preferred film forming polymer comprising at least one cyclic group selected from the group consisting of cyclic amides, cyclic amines, and mixtures thereof is LUVISET CLEAR AT3, a copolymer of N-vinyl pyrrolidone, methacrylamide, and N-vinylimidazole commercially available from BASF of Ludwigshafen, Germany.

Preferably, the film forming polymer comprising at least one cyclic group selected from the group consisting of cyclic amides, cyclic amines, and mixtures thereof is present in an amount ranging from about 1% to about 40% by weight, preferably from about 3% to about 35% by weight, preferably from about 5% to about 30% by weight, and preferably from about 7% to about 25% by weight, all weights being based on the total weight of the composition, including all ranges and subranges therebetween. Preferably, the compositions of the present invention contain 7% or more by weight of the film forming polymer comprising at least one cyclic group selected from the group consisting of cyclic amides, cyclic amines, and mixtures thereof, preferably 10% or more by weight, preferably 15% or more by weight, all weights being based on the weight of the composition.

Optionally, compositions of the present invention may further comprise at least one additional film forming agent in addition to the film forming polymer comprising at least one cyclic group selected from the group consisting of cyclic amides, cyclic amines, and mixtures thereof other groups in addition to the cyclic amide groups and/or cyclic amine groups.

If present, the at least one additional film forming agent may be any other film forming agent suitable for use in a composition for application to hair, eyebrows and/or eyelashes.

For example, the at least one additional film forming agent may include at least one cyclic amide monomer (hereinafter “second cyclic amide monomer”). The fraction (e.g., weight fraction) of the second cyclic amide monomer in the at least one additional film forming polymer is at least about 70%, preferably at least about 75%, and preferably at least about 80%. Further, the second cyclic amide monomer is preferably selected from a vinyl pyrrolidone (a γ-lactam) a caprolactam, and combinations thereof.

Suitable examples of the additional film forming agent include, but are not limited to, vinyl pyrrolidone/vinyl acetate copolymers having at least 70% vinyl pyrrolidone monomer, such as LUVIKSOL 73E, LUVIKSOL 73W; polyvinylcaprolactam, such as LUVIKSOL Plus; and polyvinyl pyrrolidone homopolymer such as PVP K-60 (or PLASDONE K-60), PVPK-90 (or PLASDONE K-90), or PVP K-120 (or PLASDONE K-120), each commercially available from Ashland, Inc. of Kovington, Ky.

Specific examples also include, but are not limited to, a silicone polymer such as, for example, a non-ionic silicone copolymer such as a non-ionic dimethicone copolymer. The silicone polymer or copolymer may be in the form of particles dispersed in an aqueous dispersion medium. Non-limiting examples of non-ionic silicone polymers include polymethylsiloxane resin, a linear block copolymer, and a mixture thereof. More specifically, non-limiting examples include a dimethicone copolymer such as a copolymer of dimethylpolysiloxane and vinyl dimethylpolysiloxane (i.e., a polydimethylsiloxane/vinyl copolymer) or a copolymer of dimethylpolysiloxane and a (meth)acrylate, with the dimethicone copolymer optionally being crosslinked and/or end-capped with functional groups. For example, a polydimethylsiloxane and vinyl dimethylpolysiloxane may comprise dimethylpolysiloxane that is crosslinked with vinyl dimethylpolysiloxane and/or dimethylpolysiloxane that is end-capped with vinyl dimethylpolysiloxane. A preferred compound includes dimethylpolysiloxane crosslinked with vinyl dimethylpolysiloxane. An example of a particularly useful dimethicone copolymer is a divinyl-dimethicone/dimethicone copolymer available as DOWSIL HMW 2220 Non-Ionic Emulsion, available from Dow Corning of Midland, Mich. This is a 60 percent active aqueous dispersion of divinyldimethicone/dimethicone copolymer and comprising C₁₂-C₁₃ Pareth-3 and C₁₂-C₁₃ Pareth-23.

Specific examples further include non-crosslinked acrylate and acrylic co-polymers, urethane polymers, polyesters and combinations thereof. A non-limiting example of a suitable non-crosslinked additional film forming agent is sodium alginate, available as PROTANAL PH 6160 from FMC Health and Nutrition of Philadelphia, Pa.

Preferably, if present, the additional film forming agent is present in an amount ranging from about 0.1% to about 40% by weight, preferably from about 0.5% to about 30% by weight, preferably from about 1% to about 20% by weight, and preferably from about 2% to about 10% by weight, all weights being based on the total weight of the composition, including all ranges and subranges therebetween. Preferably, the compositions of the present invention contain 2% or less by weight of the additional film forming agent, preferably 1% or less by weight, preferably 0.5% or less by weight, all weights being based on the weight of the composition.

Preferably, the compositions of the present invention contain more film-forming polymer comprising at least one cyclic group selected from the group consisting of cyclic amides, cyclic amines, and mixtures thereof than additional film forming agent by weight. Preferably, the film-forming polymer comprising at least one cyclic group selected from the group consisting of cyclic amides, cyclic amines, and mixtures thereof constitutes at least 55% percent by weight of the film forming component of the compositions of the present invention, at least 60% by weight, at least 75% by weight, at least 80% by weight, and least 90% by weight, all weights being based on the total weight of the film forming component of the composition, with the remainder of the film forming component being made up of the additional film forming agent(s).

For example, the film-forming component may comprise from about 60% to about 95% by weight of a copolymer of N-vinyl pyrrolidone, methacrylamide, and N-vinylimidazole; from about 9% to about 25% by weight of a nonionic film-forming polymer having a fraction of a second cyclic amide monomer that is at least about 70% (e.g., a vinyl pyrrolidone/vinyl acetate copolymer or a polyvinylcaprolactam), and optionally from about 1% to about 3% by weight of a dimethicone copolymer.

Preferably, the film forming component of the compositions of the present invention are present in an amount ranging from about 0.1% to about 40% by weight, preferably from about 0.5% to about 30% by weight, preferably from about 1% to about 25% by weight, and preferably from about 2% to about 20% by weight, all weights being based on the total weight of the composition, including all ranges and subranges therebetween. Preferably, the compositions of the present invention contain 40% or less by weight of the film forming component, preferably 30% or less by weight, preferably 25% or less by weight, all weights being based on the weight of the composition.

According to the present invention, compositions of the present invention comprise water. Preferably, water is present in the compositions of the present invention in an amount ranging from about 30% to about 90% by weight, preferably from about 40% to about 85% by weight, preferably from about 45% to about 80% by weight, and preferably from about 50% to about 75% by weight, all weights being based on the total weight of the composition, including all ranges and subranges therebetween.

According to preferred embodiments, compositions of the present invention may optionally further comprise at least one C2-C5 alcohol. Preferred examples of C2-C5 alcohols include C2-C5 monoalcohols such as ethanol, butanol, propanol or isopropanol.

Preferably, if present, the C2-C5 alcohol(s) are present in the compositions of the present invention in an amount ranging from about 0.1% to about 10% by weight, preferably from about 0.5% to about 8% by weight, preferably from about 0.75% to about 7.5% by weight, and preferably from about 1% to about 6% by weight, all weights being based on the total weight of the composition, including all ranges and subranges therebetween.

However, according to embodiments of the present invention, compositions of the present invention are free of polyhydric alcohols, essentially free of polyhydric alcohols or substantially free of polyhydric alcohols. “Polyhydric alcohols” include compositions containing two or more alcohol groups such as glycerin or glycols such as propylene, butylene or hexylene glycol.

According to preferred embodiments, compositions of the present invention may optionally further comprise at least one wax. As used herein, “wax” is intended to mean a lipophilic fatty compound that is solid at room temperature (about 25° C.) and atmospheric pressure (760 mmHg, i.e., 105 Pa), which undergoes a reversible solid/liquid change of state and which has a melting point of greater than 30° C., and in some embodiments, greater than about 55° C. up to about 120° C. or even as high as about 200° C.

“Wax” may include waxes of animal origin, waxes of plant origin, waxes of mineral origin and waxes of synthetic origin. Examples of waxes of animal origin include beeswaxes, lanolin waxes and Chinese insect waxes. Examples of waxes of plant origin include rice waxes, carnauba wax, candelilla wax, ouricurry wax, cork fiber waxes, sugar cane waxes, Japan waxes, sumach wax and cotton wax. Examples of waxes of mineral origin include paraffins, microcrystalline waxes, montan waxes and ozokerites. Examples of waxes of synthetic origin include polyolefin waxes, e.g., polyethylene waxes, waxes obtained by Fischer-Tropsch synthesis, waxy copolymers and their esters, and silicone and fluoro waxes.

“Wax” may further include high melting point hydrogenated oils of animal or plant origin. Examples include hydrogenated jojoba waxes and hydrogenated oils which are obtained by catalytic hydrogenation of fats composed of a C₈-C₃₂ linear or nonlinear fatty chain, hydrogenated sunflower oil, hydrogenated castor oil, hydrogenated copra oil, hydrogenated lanolin and hydrogenated palm oils.

Preferably, if present, the waxe(s) are present in the compositions of the present invention in an amount ranging from about 0.1% to about 20% by weight, preferably from about 0.5% to about 15% by weight, preferably from about 1% to about 10% by weight, and preferably from about 1% to about 5% by weight, all weights being based on the total weight of the composition, including all ranges and subranges therebetween.

However, according to embodiments of the present invention, compositions of the present invention are free of waxes, essentially free of waxes or substantially free of waxes.

According to preferred embodiments, compositions of the present invention may optionally further comprise at least one oil. As used herein, by “oils,” it is meant compounds having a melting point of less than about 30° C. and generally insoluble in water and includes a hydrophobic moiety, such as one meeting one or more of the following three criteria: (a) has a carbon chain of at least six carbons in which none of the six carbons is a carbonyl carbon or has a hydrophilic moiety (defined below) bonded directly to it; (b) has two or more alkyl siloxy groups; or (c) has two or more oxypropylene groups in sequence. The hydrophobic moiety may include linear, cyclic, aromatic, saturated or unsaturated groups. The hydrophobic compound is in certain embodiments not amphiphilic and, as such, in this embodiment does not include hydrophilic moieties, such as anionic, cationic, zwitterionic, or nonionic groups, that are polar, including sulfate, sulfonate, carboxylate, phosphate, phosphonate, ammonium, including mono-, di-, and trialkylammonium species, pyridinium, imidazolinium, amidinium, poly(ethyleneiminium), ammonioalkylsulfonate, ammonioalkylcarboxylate, amphoacetate, and poly(ethyleneoxy)sulfonyl moieties. In certain embodiments, the oil does not include hydroxyl moieties.

Suitable examples of compounds of oils include vegetable oils (glyceryl esters of fatty acids, triglycerides) and fatty esters. Specific non-limiting examples include, without limitation, esters such as isopropyl palmitate, isopropyl myristate, isononyl isonanoate C₁₂-C₁₅ alkyl benzoates, caprylic/capric triglycerides, silicone oils (such as dimethicone and cyclopentasiloxane), pentaerythritol tetraoctanoate and mineral oil. Other examples of oils include liquid organic ultraviolet filter commonly used for example as UV-absorbing sunscreens such as octocrylene, octyl salicylate, octyl methoxyxcinnamate, among others.

Preferably, if present, the oil(s) are present in the compositions of the present invention in an amount ranging from about 0.1% to about 20% by weight, preferably from about 0.5% to about 15% by weight, preferably from about 1% to about 10% by weight, and preferably from about 1% to about 5% by weight, all weights being based on the total weight of the composition, including all ranges and subranges therebetween.

However, according to embodiments of the present invention, compositions of the present invention are free of oils, essentially free of oils or substantially free of oils.

However, according to embodiments of the present invention, compositions of the present invention are free of both oils and waxes, essentially free of both oils and waxes or substantially free of both oils and waxes.

According to preferred embodiments, compositions of the present invention may optionally further comprise at least one surfactant or dispersant, for example to assist in wetting or dispersing particulate matter in the composition. Any surfactants, including anionic, nonionic, amphoteric, and cationic, surfactants, may be used in the present invention, as long as the surfactant is cosmetically or dermatologically acceptable. The surfactant may be used either singly or in combination two or more thereof. In one embodiment, the mascara composition may include an anionic surfactant/dispersant such as sodium laureth sulfate.

Preferably, if present, the surfactant(s) or dispersant(s) are present, individually or collectively, in the compositions of the present invention in an amount ranging from about 0.1% to about 10% by weight, preferably from about 0.5% to about 8% by weight, preferably from about 0.75% to about 6% by weight, and preferably from about 1% to about 5% by weight, all weights being based on the total weight of the composition, including all ranges and subranges therebetween.

However, according to embodiments of the present invention, compositions of the present invention are free of surfactant(s) and dispersant(s), essentially free of surfactant(s) and dispersant(s) or substantially free of surfactant(s) and dispersant(s).

According to preferred embodiments, compositions of the present invention may optionally further comprise at least one particulate.

Suitable colorants include, but are not limited to inorganic particulates that impart color or optical effects and organic pigments. Particulate materials are generally finely divided particulates that are insoluble in but are otherwise homogeneously stabilized (suspended or dispersed) in a vehicle of the composition. Preferably, particulate materials are materials that are incapable of chemically “self-fusing” in-use and are not themselves film-forming.

Suitable inorganic particulate materials include any of a variety of porous, semi-porous, non-porous, or hollow, coated or uncoated water-insoluble inorganic particulates such as silica, alumina, carbon and any of various oxides, silicates, aluminosilicates, nitrides, carbides, carbonates, and the like. In particular embodiments, the inorganic particulate is selected from carbon black, silica, and iron oxide. Other particulates, e.g., organic pigments such as lake pigments. Other organic particulates such as polymeric particulates including nylon particulates, acrylate particulates (e.g., PMMA), silicone elastomer particulates, and the like may also be used.

Preferably, if present, the particulate(s) (for example, colorants such as pigments) are present in the compositions of the present invention in an amount ranging from about 0.1% to about 20% by weight, preferably from about 0.5% to about 15% by weight, preferably from about 1% to about 12% by weight, and preferably from about 1% to about 10% by weight, all weights being based on the total weight of the composition, including all ranges and subranges therebetween.

According to preferred embodiments, compositions of the present invention may optionally further comprise at least one dye. Any of various lipophilic or water soluble dyes may be used as well.

Preferably, if present, the dye(s) are present in the compositions of the present invention in an amount ranging from about 0.1% to about 20% by weight, preferably from about 0.5% to about 15% by weight, preferably from about 1% to about 12% by weight, and preferably from about 1% to about 10% by weight, all weights being based on the total weight of the composition, including all ranges and subranges therebetween.

Typically, when the composition contains colorants (dyes and/or particulate colorants), the composition may be used as a mascara composition. Alternatively, when the composition does not contain colorants, it is a clear or transparent composition which can be used as a basecoat (or topcoat) prior to (or after) application of a mascara composition to keratinous materials. A composition free of colorants may also be used as a solitary coating (without an additional separate basecoat or topcoat). However, it is possible that topcoats or basecoats could contain colorants, and/or that a mascara composition could contain little or no colorant.

In order to facilitate application to the hair, eyebrows and/or eyelashes, compositions of the present invention generally include a vehicle in which the film-forming polymer portion is stabilized (i.e., dissolved, dispersed or suspended). The vehicle generally includes, consists of or consists essentially of water.

The compositions of the present invention may optionally further include various additives desirably used in cosmetic or dermatological compositions. For example, anti-oxidants, pH adjusters, preservatives, neutralizing agents, fragrances, plasticizers, cosmetic and dermatological active agents such as emollients, moisturizers, vitamins, UV filters, and mixtures thereof can be added. A non-exhaustive listing of such ingredients can be found in the CTFA International Cosmetic Ingredient Dictionary and Handbook, Fourteenth Edition (2012), contents of which are incorporated herein by reference in its entirety.

One of ordinary skill in the art can select optional additional additives and/or the amount thereof such that the advantageous properties of the compositions according to the present invention are not, or are not substantially, adversely affected by the envisaged addition.

Needless to say, the composition of the invention should be cosmetically or dermatologically acceptable, i.e., it should contain a non-toxic physiologically acceptable medium and should be able to be applied to the eyelashes of human beings.

The compositions of the present invention are intended to be applied onto hair, eyelashes and/or eyebrows. In preferred embodiments, the composition is a mascara and it is applied to a portion of eyelash(es) surface to which one desires to impart a concave curvature, such as a curling effect on the eyelash(es).

In this regard, the inventors have found that compositions of the present invention are useful for self-curling of eyelash(es) by applying them to the surface of eyelash(es) in which the curling effect is desired and allowing the composition to dry. So, for example, applying the composition to the top surface of the upper eyelash results in an upward curl of the top eyelash.

Accordingly, one aspect of the present invention are methods of making up hair, eyebrows and/or eyelashes comprising applying compositions of the present invention to the hair, eyebrows and/or eyelashes using the applicator of the present invention in an amount sufficient to make up the hair, eyebrows and/or eyelashes and allowing the composition to dry. Without wishing to be bound by any theories, it is believe that the compositions of the present invention, by virtue of their ingredients, result in film formation on the hair, eyebrows and/or eyelashes which shrinks during drying, thereby resulting in a self-curling effect in the direction oriented toward the surface of the hair, eyebrow and/or eyelash to which the composition was applied.

Accordingly, one aspect of the present invention are methods of curling hair, eyebrows and/or eyelashes comprising applying compositions of the present invention to the hair, eyebrows and/or eyelashes in an amount sufficient to curl the hair, eyebrows and/or eyelashes using the applicator of the present invention and allowing the composition to dry. Without wishing to be bound by any theories, it is believe that the compositions of the present invention, by virtue of their ingredients, result in film formation on the hair, eyebrows and/or eyelashes which shrinks during drying, thereby resulting in a self-curling effect in the direction oriented toward the surface of the hair, eyebrow and/or eyelash to which the composition was applied.

In accordance with the above methods, the compositions of the present invention may be applied to hair, eyebrows and/or eyelashes as needed, preferably once or twice daily, more preferably once daily. As noted above, the compositions should be allowed to dry after application.

Compositions of the present invention may be made by mixing at least one film-forming polymer comprising at least one cyclic group selected from the group consisting of cyclic amides, cyclic amines, and mixtures thereof in water until dissolution. This film forming polymer and other water-soluble ingredients may be mixed by stirring, shaking, grounding, or beating, optionally with a stirrer, a magnetic stirrer, a shaker, a homogenizer, or any other methods suitably used to mix cosmetic composition. The mixing may be carried out with or without heating or cooling the ingredients. Particulates and other ingredients that are to be dispersed are then added with mixing to form a homogeneous mixture.

According to embodiments of the present invention, compositions of the present invention may be used as a “primer” (first treatment in a two-step process) such as before using as a second step a traditional composition such as a mascara (or other hair, eyebrow and/or eyelash composition). The second step may include applying the traditional composition (e.g., mascara) to the opposite surface of the hair, eyebrows and/or eyelashes to which the invention composition has been applied. However, the traditional composition may also be applied to the same surface of the hair, eyebrows and/or eyelashes that the compositions of the present invention have been applied. Also, the traditional composition can be applied to both surfaces of the hair, eyebrows and/or eyelashes, if desired.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

### Example 1—Evaluation of Applicators

Four different applicators were evaluated by consumers. They were each tested using an identical composition. The composition included water, LUVISET CLEAR AT3, a non-ionic water-soluble copolymer that includes a cyclic amide monomer, a cyclic amine monomer, and an acrylamide monomer (BASF Corporation). The composition further included SIMULGEL 600 (Seppic, Inc. of Fairfield, N.J.), PROTANAL PH 6160 (FMC BioPolymer), carbon black and water (q.s.). The composition included about had a viscosity of about 161 Pa S when measured at a shear rate at 5 s⁻¹.

The four applicators were commercially available. The first of the four, Inventive Example 1, was a mascara applicator commercially available from Geka Manufacturing Company, a subsidiary of Sulzer Ltd. of Winterthur, Switzerland which had 1248 total bristles and a surface area of around 260 mm², resulting in around 4.8 bristles per mm², and had a high density zone that covers at least a portion of the outer surface of the core, the high density zone having a surface bristle density of up to 7 bristles per square millimeter of area of the outer surface of the core, and wherein the bristles had a length from about 0.6 mm to about 4 mm.

The second, Comparative Example 1 was a flocked fiber applicator not typically used for mascara. It did not have a high density zone having a surface bristle density of up to 7 bristles per square millimeter of area (the bristle density was considerably greater).

The third, Comparative Example 2 was a conventional fiber-based mascara straight applicator formed from twisted/trimmed fibers. It did not have a high density zone having a surface bristle density of up to 7 bristles per square millimeter of area (as with the Comparative Example 1, the bristle density was considerably greater).

The fourth, Comparative Example 3 was a conventional fiber-based mascara curved applicator formed from twisted/trimmed fibers. It did not have a high density zone having a surface bristle density of up to 7 bristles per square millimeter of area (as with the other Comparative Examples, the bristle density was considerably greater).

The applicators were evaluated for their ability to successfully apply the composition to consumers' eyelashes. Specifically, each of nine make-up artists applied the composition separately using each of the four applicators to the tops of their lashes. Each make-up artist was then asked to rate the experience by whether they agreed or disagreed with eleven of various statements/attributes.

None of the applicators of Comparative Examples 1-3 had a high density zone that covers at least a portion of the outer surface of the core, the high density zone having a surface bristle density of up to 7 bristles per square millimeter of area of the outer surface of the core, and wherein the bristles have a length from about 0.6 mm to about 4 mm.

For Inventive Example 1, ten of eleven attributes were positive and one was mixed. The make-up artists all agreed that their initial reaction was positive, the experience was differentiated from conventional mascara, the applicator was intuitive to use on the top lash, provided even application, provided control, did not transfer to lids, separated lashes/did not clump, the brush easily glided through the lashes, the gesture was easy, and the end look was positive with respect to curl, lift, and separation. Only the response to whether it deposited the right amount of product was mixed (some positive/negative).

For Comparative Example 1, the application was only rated positive in five of the eleven attributes (positive initial reaction, differentiated from conventional mascara, intuitive to use on top lash, right amount of product deposited, and easy gesture) mixed in two (even application and end look was positive with respect to curl, lift, and separation), and negative in the rest.

For Comparative Example 2, the application was rated positive in zero of the eleven attributes, mixed in three (positive initial reaction, differentiated from conventional mascara, and provided control), and negative in the rest.

For Comparative Example 3, the application was rated positive in zero of the eleven attributes, mixed in two (positive initial reaction, differentiated from mascara), and negative in the rest.

The results show that when used with a composition having a viscosity of less than about 250 Pa S when measured at a shear rate at 5 s⁻¹, the applicator having a high density zone that covers at least a portion of the outer surface of the core, the high density zone having a surface bristle density of up to 7 bristles per square millimeter of area of the outer surface of the core, where the bristles have a length from about 0.6 mm to about 4 mm, performed surprisingly well in evaluations with consumers.

### Example 2—Testing of Composition Properties

Mascara compositions were prepared by mixing into water various ingredients: a film forming polymer portion, and various functional ingredients (thickener, preservative, pigment, antioxidant).

A shear rate viscosity sweep (ramping 0.01/s-1000/s shear rate) was performed using a Discovery HR-3 rheometer from TA Instruments of New Castle, Del. to determine viscosity as a function of shear rate. The viscosity at a shear rate of 5 s⁻¹ is reported in Table 1 below.

The following SELF-CURLING TEST was also performed on the various compositions. Using a flat iron, hair fiber strands (fake eyelashes), 12 mm in length secured between metal plates—available from SP Equation of Pourcieux, France—were straightened by gently stroking them using a commercially available hair straightener set to 450° F. for a sufficient period to straighten the fibers. A mascara applicator of Inventive Example 1 commercially available from Geka Manufacturing Company, a subsidiary of Sulzer Ltd. of Winterthur, Switzerland was used to apply compositions to the hair fiber strands. The applicator had a high density zone that covers at least a portion of the outer surface of the core, the high density zone having a surface bristle density of up to 7 bristles per square millimeter of area of the outer surface of the core, and wherein the bristles have a length from about 0.6 mm to about 4 mm.

The various compositions to be tested were applied to the fake eyelashes and stroked ten times on one side of the simulated eyelashes to deposit approximately 2 mg to 10 mg of composition. The treated lashes were put into a humidity chamber (25%-45% RH and 32° C.) for 5 minutes. A curl measurement was then taken by placing a protractor near the eyelashes and visually estimating the angle of curl relative to the horizontal surface of the metal plate within which the fake eyelashes are secured.

A summary of the compositions, the results of the rheometer testing and SELF-CURLING TEST are shown in Table 1.

TABLE 1

| | | Film-Forming | Viscosity @ | |
|---|---|---|---|---|
| | | Polymers | Shear Rate of | Self-Curl |
| Ref. | Film-Forming Polymers | (wt. %) | 5 s⁻¹ Pa · S | (degrees) |
| Inventive | copolymer of N-vinyl pyrrolidone, and | 20% | 161 | 45 |
| Example 1 | methacrylamide, and N- | | | |
| | vinylimidazole¹ and sodium alginate² | | | |
| Comparative | polyvinyl pyrrolidone homopolymer³ | 33% | 262 | 15 |
| Example 1 | and ethyacrylate/methylacrylic acid/ | | | |
| | methacrylamide/methacrylic acid | | | |
| | ester of ethoxylated fatty alcohol | | | |
| | crosslinked copolymer⁴ and | | | |
| | acrylamide/acrylamidopropanessulfonate/ | | | |
| | methacrylates terpolymer⁵ and sodium | | | |
| | alginate² | | | |
| Comparative | polyvinyl pyrrolidone homopolymer³ | 30% | 1410 | <10 |
| Example 3 | and acrylates copolymer⁶ | | | |
| Inventive | polyvinyl pyrrolidone homopolymer³ | 30% | 145 | 25-40 |
| Example 2 | and ethyacrylate/methylacrylic acid/ | | | |
| | methacrylamide/methacrylic acid | | | |
| | ester of ethoxylated fatty alcohol | | | |
| | crosslinked copolymer⁴ and | | | |
| | acrylamide/acrylamidopropanessulfonate/ | | | |
| | methacrylates terpolymer⁵ and sodium | | | |
| | alginate² | | | |

¹LUVISET CLEAR AT3, a copolymer of N-vinyl pyrrolidone, methacrylamide, and N-vinylimidazole from BASF of Ludwigshafen, Germany
²PROTANAL PH 6160 from FMC Health and Nutrition of Philadelphia, PA
³PVP K-120 from Ashland, Inc. of Kovington, KY
⁴LUVISET ONE from BASF of Ludwigshafen, Germany
⁵ACUDYNE SCP from Dow Chemical of Midland, Michigan
⁶LUVIMER MAE 6160 from BASF of Ludwigshafen, Germany

The results indicate that when applied with an applicator having a high density zone that covers at least a portion of the outer surface of the core, the high density zone having a surface bristle density of up to 7 bristles per square millimeter of area of the outer surface of the core, and wherein the bristles have a length from about 0.6 mm to about 4 mm, self-curling is vastly improved when using a composition having a viscosity at a shear Rate of 5 s⁻¹ that is less than about 250 Pa S. Together with the data in Example 1 it is clear that both the applicator and the composition are important to achieving the desirable self-curling.

What is claimed is:

1. A kit comprising:
an applicator comprising a brush comprising a core having an outer surface and a longitudinal axis;
a plurality of bristles protruding from the core and spaced apart along the longitudinal axis; and
a high density zone that covers at least a portion of the outer surface of the core, the high density zone having a surface bristle density of up to 7 bristles per square millimeter of area of the outer surface of the core, and wherein the bristles have a length from about 0.6 mm to about 4 mm; and
a cosmetic composition for application to hair, eyebrows and/or eyelashes having a viscosity of less than about 250 Pa·S when measured at a shear rate at 5 s⁻¹

2. The kit according to claim 1, wherein the high density zone has a surface bristle density of 2 to 7 bristles per square millimeter of area of the outer surface of the core.

3. The kit according to claim 1, wherein the high density zone has a surface bristle density of 4 to 6 bristles per square millimeter of area of the outer surface of the core.

4. The kit according to claim 1, wherein the bristles are made of a thermoplastic elastomer.

5. The kit according to claim 1, wherein the cosmetic composition is a mascara.

6. The kit according to claim 1, wherein the cosmetic composition comprises water and a film forming component comprising at least one film-forming polymer comprising at least one cyclic group selected from the group consisting of cyclic amides, cyclic amines, and mixtures thereof.

7. The kit according to claim 6, wherein the film forming component composition comprises further comprises at least one additional film forming agent.

8. The kit according to claim 6, wherein film forming component is present in the cosmetic composition in an amount of 40% or less by weight based on the weight of the cosmetic composition.

9. The kit according to claim 7, wherein film forming component is present in the cosmetic composition in an amount of 40% or less by weight based on the weight of the cosmetic composition.

10. The kit according to claim 6, wherein film forming component is present in the cosmetic composition in an amount of 30% or less by weight based on the weight of the cosmetic composition.

11. The kit according to claim 7, wherein film forming component is present in the cosmetic composition in an amount of 30% or less by weight based on the weight of the cosmetic composition.

12. The kit according to claim 6, wherein the cosmetic composition is substantially free of oils and waxes.

13. The kit according to claim 6, wherein the cosmetic composition is free of oils and waxes.

14. The kit according to claim 6, where the cosmetic composition further comprises at least one C2-C5 alcohol.

15. The kit according to claim 14, wherein the C2-C5 alcohol is selected from the group consisting of ethanol, isopropanol, and mixtures thereof.

16. A method of curling eyelashes comprising applying the cosmetic composition of claim 1 to eyelashes in an amount sufficient to curl eyelashes using the applicator of claim 1.

17. A method of making up eyelashes comprising applying the cosmetic composition of claim 1 to eyelashes in an amount sufficient to make up eyelashes using the applicator of claim 1.
